(19) United States
(12) Patent Application Publication
(10) Pub. No.: US 2015/0182423 A1
(43) Pub. Date: Jul. 2, 2015

(54) ANTIPERSPIRANT APPLICATION DEVICE INCLUDING A SKIN-ADHESIVE PATCH AND METHODS OF USING THE SAME

(71) Applicant: The Dial Corporation, Scottsdale, AZ (US)

(72) Inventors: Catherine Schmit, Glendale, AZ (US); Debra K. Butterworth, Scottsdale, AZ (US); Jennifer Harris, Phoenix, AZ (US); Robert Demson, Cave Creek, AZ (US)

(21) Appl. No.: 14/144,662

(22) Filed: Dec. 31, 2013

(51) Int. Cl. A61K 8/02; A61K 8/26; A61K 8/27; A61Q 15/00; A61K 8/28

(52) U.S. Cl. CPC A61K 8/0208; A61Q 15/00; A61K 8/28; A61K 8/27; A61K 8/26

(57) ABSTRACT

Antiperspirant application devices including antiperspirant compositions and methods of using the antiperspirant application devices are provided herein. In an embodiment, an antiperspirant application device includes a skin-adhesive, transdermal patch including an adhesive compound capable of adhesion to human skin and an antiperspirant active compound based upon an astringent metallic salt disposed within at least a portion of the skin-adhesive, transdermal patch.

### TECHNICAL FIELD

The present disclosure generally relates to antiperspirant compositions. More particularly, the present disclosure relates to adhesive patches including solubilized antiperspirant compositions for adhering to the skin.

### BACKGROUND

Antiperspirants are popular personal care products used to prevent or eliminate perspiration and body odor caused by perspiration. Antiperspirant products, including for example sticks, emulsions, aerosol sprays, and roll-on antiperspirants are desired by a large majority of the population because of the presence of active antiperspirant compounds that minimize or prevent the secretion of perspiration by blocking or plugging ducts of sweat-secreting glands, such as those located at the underarms. Antiperspirants typically include an active antiperspirant compound in a carrier that permits the antiperspirant product to be applied to the skin by swiping or rubbing the stick across the skin, typically of the underarm. Upon application of the antiperspirant product, the carrier coats the skin or evaporates, releasing the active antiperspirant compound from the antiperspirant product upon exposure to moisture to form plugs in the sweat ducts.

Active antiperspirant compounds reduce underarm wetness and odor by migrating into openings of the sweat gland ducts and reacting with proteins therein to form antiperspirant plugs, which mechanically prevent sweat from escaping the ducts. Two types of sweat glands are present in the underarm region. The first type of sweat gland, the apocrine sweat gland, terminates and secretes at the top of hair follicles. As such, active antiperspirant compounds should migrate into the hair follicle to access the apocrine sweat gland duct and block secretion. The second type of sweat gland, the eccrine sweat gland, opens directly onto the skin. Eccrine sweat is responsible for the largest volume of sweat that causes underarm wetness. As with apocrine sweat glands, active antiperspirant compounds migrate into the eccrine sweat gland openings and form plugs, which reduce underarm wetness.

Some antiperspirant compositions known in the art of the stick type include one or more structurant compounds, which are included to provide good “glide” qualities (i.e., ease of application when the stick is moved across the underarm region) and to act as a carrier of the antiperspirant active ingredient at the declared percent U.S.P. dosage. Other antiperspirant compositions known in the art include emollients and waxes to improve “skin feel” upon application of the antiperspirant. However, these structurants, emollients, and waxes leave a residue on the skin that can create a barrier against the migration of the active antiperspirant compounds into the sweat glands, thus potentially reducing the effectiveness of the antiperspirant composition.

Accordingly, it is desirable to provide antiperspirant compositions that exhibit improved migration into the sweat glands. Additionally, it is desirable to provide antiperspirant application products and methods that do not require the use of structurants, emollients, and waxes. Furthermore, other desirable features and characteristics of the present invention will become apparent from the subsequent detailed description of the invention and the appended claims, taken in conjunction with the accompanying drawings and this background of the invention.

### BRIEF SUMMARY

Antiperspirant application devices including antiperspirant compositions and methods of using the antiperspirant application devices are provided herein. In an exemplary embodiment, an antiperspirant application device includes a skin-adhesive, transdermal patch including an adhesive compound capable of adhesion to human skin and an antiperspirant active compound based upon an astringent metallic salt disposed within at least a portion of the skin-adhesive, transdermal patch.

In another exemplary embodiment, a method of applying an antiperspirant active compound to the skin includes the step of applying to the skin an antiperspirant application device including a skin-adhesive, transdermal patch including an adhesive compound capable of adhesion to human skin and an antiperspirant active compound based upon an astringent metallic salt disposed within at least a portion of the skin-adhesive, transdermal patch. The method further includes the steps of leaving the antiperspirant application device in place on the skin for a period of time and removing the antiperspirant application device from the skin.

This brief summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWING

Embodiments of the present disclosure will hereinafter be described in conjunction with the following drawing figure, wherein like numerals thereof denote like elements, and wherein:

FIG. 1 is an exemplary skin-adhesive patch containing an active antiperspirant composition in accordance with various embodiments of the present disclosure.

### DETAILED DESCRIPTION

The following detailed description is merely exemplary in nature and is not intended to limit the invention or the application and uses of the invention. Furthermore, there is no intention to be bound by any theory presented in the preceding background or the following detailed description.

Embodiments of the present disclosure are generally directed to antiperspirant compositions and adhesive skin patches containing antiperspirant compositions. Current antiperspirant compositions are traditionally applied to the skin using a stick, gel, roll-on, or soft solid. The formation of antiperspirant plugs in eccrine sweat glands of the skin, particularly in the underarm region, is a result of the dissolutions of the antiperspirant active ingredient into eccrine sweat and migration deeper into sweat glands. Upon migration into the glands, the active antiperspirant composition gels and form plugs which inhibit sweat from reaching skin.

Embodiments of the present disclosure use the same principle of migration of the antiperspirant active compounds into the skin, except that the active antiperspirant composition is applied to the skin using an adhesive patch, applied to the underarm region, that contains the active antiperspirant composition. This adhesive patch contains the antiperspirant active in a pre-solubilized form and puts it in direct contact with the underarm skin. The occlusion provided by the patch would keep skin moist, and the antiperspirant active diffuses out of the patch along this moisture to the source, namely the ecrrine gland. This action allows salt plugs to be efficiently formed in the axilla region during while the patch remains in place adhered to the underarm region. In this manner, it is not necessary for the antiperspirant composition formula to use emollients, waxes, and structurants that are used in typical forms, which leave a residue on the skin that can create a barrier to the active antiperspirant compounds, slowing down the diffusion thereof into the eccrine glands.

In practice, a flexible self-adhesive patch is impregnated with an aqueous, hydro-alcoholic, or glycolic, or mixtures thereof, antiperspirant active solution. The ergonomic antiperspirant patch is applied to the axilla before a period of rest, or at any other time. The patch is left in place for several hours allowing for antiperspirant salt plug formation to occur. The patches are removed after a period of time leaving no residue, and the antiperspirant protection is then active. The described embodiments allow the user to apply the antiperspirant patch hours before protection is needed, for example at night, to have efficient delivery of the antiperspirant active compound into the eccrine glands to block sweat, and then remove the patch for day time activities, for example.

The disclosure that follows provides exemplary antiperspirant active compositions, exemplary adhesive skin patches that contain such compounds, and exemplary methods of use of such adhesive skin patches.

### Antiperspirant Active Composition

The antiperspirant active compound is based upon an astringent metallic salt. By “based upon”, it is meant that the antiperspirant active compound is either an astringent metallic salt, or is derived from an astringent metallic salt (such as a complex of an astringent metallic salt, buffer, and water). The antiperspirant active compound may be introduced into the antiperspirant composition through an antiperspirant active component that includes a separate buffer in addition to the antiperspirant active compound. Antiperspirant active compounds based upon astringent metallic salts are known in the art.

Examples of suitable antiperspirant active compounds include astringent salts of aluminum, zirconium, zinc, and mixtures thereof, which may be inorganic or organic salts. Some examples of inorganic and organic aluminum-containing and/or zirconium-containing salts include aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b}x(H₂O), wherein a is from 2 to about 5; the sum of a and b is about 6; x is from about 1 to about 6; and wherein a, b, and x may have non-integer values. Exemplary zirconium salts include those having the general formula ZrO(OH)₂₋ₐClₐx(H₂O), wherein a is from about 1.5 to about 1.87, x is from about 1 to about 7, and wherein a and x may both have non-integer values. In an embodiment, the antiperspirant active compound includes zirconium salt complexes that additionally contain aluminum and glycine, commonly known as ZAG complexes. These ZAG complexes contain aluminum chlorohydroxide and zirconyl hydroxy chloride conforming to the above-described formulas. Examples of antiperspirant active compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof.

In an embodiment, the antiperspirant active compound is a salt of aluminum and zirconium having a ratio of aluminum to zirconium of greater than 6.0:1.0, such as from about 6.0:1.0 to 10.0:1.0. For example, in this embodiment, the antiperspirant active compound may include aluminum zirconium pentachlorohydrate, aluminum zirconium pentachlorohydrex glycine complex, or a combination of the aforementioned compounds. Aluminum zirconium pentachlorohydrex glycine complexes are commercially available, for example, from Summit Reheis of Huguenot, New York under the designation AAZG-3110 as powder. In another example of this embodiment, the antiperspirant active compound may include aluminum zirconium octachlorohydrex glycine complex, commercially available, for example, from Summit Reheis under the designation AAZG-3109.

The antiperspirant active compound is present in the antiperspirant composition in an amount that provides a measurable antiperspirant effect when applied to the underarms of a consumer. In an embodiment, the antiperspirant active compound is present in an amount of from about 10 to about 25 weight % (USP), such as from about 14 to about 20 weight % (USP), based on the total weight of the antiperspirant composition. As used herein, weight % (USP) of an antiperspirant compound is calculated as anhydrous weight percent in accordance with the U.S.P. method, as is known in the art. This calculation excludes any bound water and buffer such as glycine. It is to be appreciated that most commercially available antiperspirant active compounds are sold in mixtures along with other compounds that are not considered antiperspirant active compounds, such as water and buffer as described above in the context of the antiperspirant active component, and the above values do not account for the presence of such non-active compounds that may be present in the antiperspirant active component. In an embodiment, the antiperspirant active component includes antiperspirant active compounds in an amount of from about 30 to about 99 weight %, such as from about 50 to about 85 weight %, based on the total weight of the antiperspirant active component.

As noted above, the active antiperspirant salts may be provided in the adhesive patch in a pre-solubilized form. Thus, the active antiperspirant salts may be provided solubilized in one or more solvents, such as water, glycol, or an alcohol. In an exemplary embodiment, the pre-solubilized antiperspirant active compound is based upon water and/or water-miscible compounds that are liquid at ambient temperature of about 21° C. The pre-solubilized form may thus include an antiperspirant active compound and water. Optional components may also be included in the first liquid phase such as, but not limited to, a buffer component, a monohydric alcohol component, and/or dye.

In addition to the water, and as alluded to above, the pre-solubilized form may also include a monohydric alcohol component that, together with the water, functions as a carrier solvent. Suitable monohydric alcohols that may be included are not particularly limited and may include any monohydric alcohols that are known to be included in antiperspirant compositions. In one embodiment, the monohydric alcohol component includes one or more C1 to C6 monols. Examples of suitable monohydric alcohols include, but are not limited to, methanol, ethanol, propanol, isopropanol, and combinations thereof, with ethanol being particularly preferred. In an embodiment, the monohydric alcohol component is present in an amount of from greater than about 0 to about 25 weight %, for example from about 15 to about 25 weight %, based upon the total weight of the solubilized antiperspirant composition.

The optional buffer component (also referred to as a buffering agent) represents any suitable buffers for antiperspirant active compounds as are known in the art and can include polyhydric alcohols, amino acids, and combinations thereof. The buffers function to stabilize pH of the antiperspirant active compounds and reduce skin irritability that the antiperspirant active compounds may cause. Additionally, the buffers may contribute to activation of the antiperspirant active compounds. In one embodiment, the buffer component includes one or more amino acids. Suitable amino acids include glycine, alanine, leucine, isoleucine, β-alanine, valine, cysteine, serine, tryptophane, phenylalanine, methionine, β-amino-n-butanoic acid, γ-amino-n-butanoic acid, and salts thereof, which may be in the d-form, the i-form or the dl-form. In one specific embodiment of the present disclosure, the buffer component includes the buffer compound glycine. In an embodiment, the buffer component is present in the first liquid phase in an amount of from about 0.5 to about 4.0 weight %, such as, for example, from about 0.7 to about 3.5 weight %, or, as another example, from about 2.0 to about 3.0 weight %, or as another example, about 2.5 weight %, all based upon the total weight of the solubilized active antiperspirant composition.

The antiperspirant composition contemplated herein also may include additives, such as those used in conventional antiperspirants. For example, in addition to the above-described salts and solvents, additives that cause the antiperspirant product to exhibit long-lasting fragrance, odor protection, bacteria control, and/or another desired purpose and/or function may be included. These additives include, but are not limited to, fragrances, including encapsulated fragrances, dyes, pigments, preservatives, antioxidants, moisturizers, and the like. These optional ingredients can be included in an amount of about 0 to about 20 wt. %.

### Skin Adhesive Patch

As noted above, in embodiments of the present disclosure, the active antiperspirant composition is applied to the skin using an adhesive patch, applied to the underarm region, which contains the active antiperspirant composition. This adhesive patch contains the antiperspirant active in a pre-solubilized form and puts it in direct contact with the underarm skin. A flexible self-adhesive patch is impregnated with an aqueous, glycolic, or hydro-alcoholic antiperspirant active solution. The ergonomic antiperspirant patch is applied to the axilla before a period of rest, or at any other time. The patch is left in place for several hours allowing for antiperspirant salt plug formation to occur. The patches are removed after a period of time leaving no residue, and the antiperspirant protection is then active.

Various skin adhesive or transdermal patches are known in the art, and are suitable for use with the embodiments described herein. In some embodiments, the skin adhesive patch may be embodied as a single-layer antiperspirant-in-adhesive patch. In these embodiments, the adhesive layer of this system also contains the antiperspirant active composition. In this type of patch the adhesive layer not only serves to adhere the various layers together, along with the entire system to the skin, but is also responsible for the releasing of the antiperspirant active composition. The adhesive layer is surrounded by a temporary liner and a backing.

In other embodiments, the skin adhesive patch may be embodied as a multi-layer antiperspirant-in-adhesive patch. In these embodiments, the multi-layer antiperspirant-in adhesive patch is similar to the single-layer system in that both adhesive layers are also responsible for the releasing of the antiperspirant active composition. One of the layers is for immediate release of the antiperspirant active composition and other layer is for control release of antiperspirant active composition from a reservoir. The multi-layer system is different however that it adds another layer of antiperspirant-in-adhesive, usually separated by a membrane (but not in all cases). This patch also has a temporary liner-layer and a permanent backing. The antiperspirant active composition release from this depends on membrane permeability and diffusion of antiperspirant active composition molecules, which may be suitably selected for a particular application by the skilled artisan.

In further embodiments, the skin adhesive patch may be embodied as a reservoir-type patch. Unlike the above-described single-layer and multi-layer antiperspirant-in-adhesive systems, the reservoir transdermal system has a separate antiperspirant active composition layer. The antiperspirant active composition layer is a liquid compartment containing a antiperspirant active composition solution or suspension (for example, the pre-solubilized form described above) separated by the adhesive layer. This patch is also backed by the backing layer. In these embodiments, the drug reservoir is totally encapsulated in a shallow compartment molded from antiperspirant active composition-impermeable metallic plastic laminates, and the another side of the membrane includes polymeric membrane such as ethyl vinyl acetate, for example.

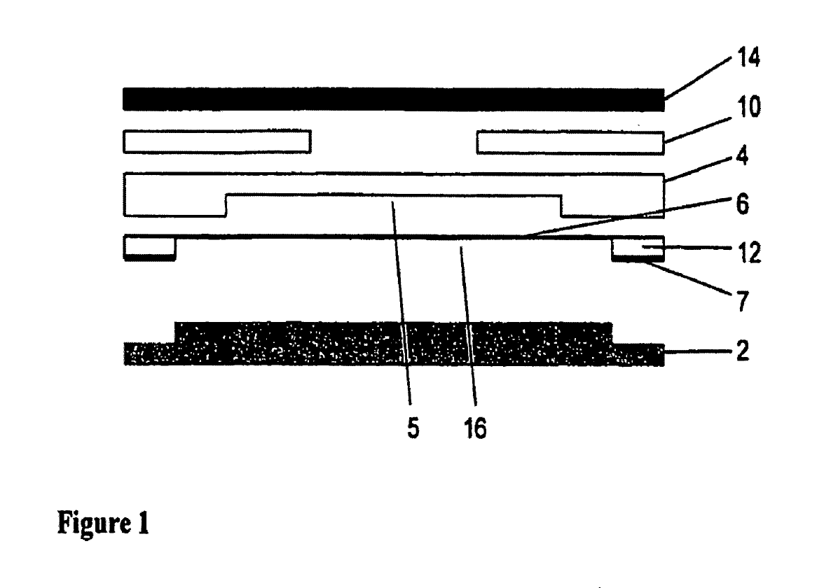

An exemplary reservoir-type patch known in the art and suitable for use with the embodiments described herein is shown in FIG. 1. Between top and bottom layers 14 and 2, respectively, a circular reservoir layer 4 includes a central reservoir chamber 5 in which the active antiperspirant composition for delivery to the skin (for example, the axilla) is stored. The active antiperspirant composition is retained in the chamber 5 by a porous resilient membrane 6, which may or may not be permeable to the active antiperspirant composition in the rest state of the patch, as described below. Around the periphery of the resilient membrane 6, a thicker ring of material 12 provides a downwardly facing surface on which is formed a layer of adhesive 7 for adhering the patch to the skin of the user. A further ring 10 may also be included. Inside the thicker ring 12 a compartment 16 is formed between the resilient membrane 6 and the skin of the user, which allows the active antiperspirant composition to collect and spread out after passing through the resilient membrane 6 and before passing into the skin.

In still further embodiments, the skin adhesive patch may be embodied as a matrix-type patch. The matrix system has an antiperspirant active composition layer of a semisolid matrix containing an antiperspirant active composition solution or suspension. The adhesive layer in this patch surrounds the antiperspirant active composition layer partially overlaying it. This type of patch is also known in the art as a monolithic device.

In accordance with one exemplary embodiment, the skin adhesive patch may include an adhesive capable of containing and delivering the active antiperspirant composition, such adhesive being prepared in accordance with the disclosure set forth in commonly-assigned United States Patent Application Publication no. 2009/0130188, titled: “Acrylic pressure sensitive adhesives,” the contents of which are incorporated by reference herein in their entirety. Of course, the use of other adhesives is possible.

### Methods of Use

An skin-adhesive antiperspirant patch, containing a pre-solubilized antiperspirant active composition according to any of the formulations described above, is provided as the active ingredient in any of the skin-adhesive patch types described above. The user applies the provided antiperspirant patch to the skin. One or more patches may be applied. For example, one patch may be applied under each arm. For example, the antiperspirant patch is applied to the axilla. The user applies the antiperspirant patch at a time preferably before it is anticipated that the antiperspirant effect is needed or desired, for example before a period of rest, or at any other time. The user leaves the patch in place for a period of time, for example from about 1 hour to about 10 hours, such as about 2 hours to about 8 hours. The patch is left in place to allow for antiperspirant salt plug formation to occur. Namely, the active antiperspirant composition migrates from the patch (in any of the above-described manners) into the skin of the user, particular into the skin of the underarm region of the user, and into the sweat glands of the user, such as the eccrine glands. After the aforesaid period of the, the patch is removed. As the active antiperspirant composition preferably contains no structurants, emollients, or waxes, the removal of the patch leaves no water-insoluble residue behind on the user's skin. At this time, the antiperspirant protection is active.

Accordingly, the described embodiments desirably provide antiperspirant compositions that exhibit improved migration into the sweat glands. Additionally, the described embodiments provide antiperspirant application products and methods that do not require the use of structurants, emollients, and waxes. Further, the described embodiments allow the user to apply the antiperspirant patch hours before protection is needed, for example at night, to have efficient delivery of the antiperspirant active compound into the eccrine glands to block sweat, and then remove the patch for day time activities, for example.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. An antiperspirant application device comprising:
a skin-adhesive, transdermal patch comprising an adhesive compound capable of adhesion to human skin; and
an antiperspirant active compound based upon an astringent metallic salt disposed within at least a portion of the skin-adhesive, transdermal patch.

2. The antiperspirant application device of claim 1, wherein the skin-adhesive, transdermal patch is a single-layer antiperspirant-in-adhesive patch.

3. The antiperspirant application device of claim 1, wherein the skin-adhesive, transdermal patch is a multi-layer antiperspirant-in-adhesive patch.

4. The antiperspirant application device of claim 1, wherein the skin-adhesive, transdermal patch is a reservoir-type patch.

5. The antiperspirant application device of claim 1, wherein the skin-adhesive, transdermal patch is a matrix-type patch.

6. The antiperspirant application device of claim 1, wherein the adhesive compound is an acrylic, pressure-sensitive adhesive that comprises, at least in part, the antiperspirant active compound.

7. The antiperspirant application device of claim 1, wherein the antiperspirant active compound is selected from the group consisting of: salts of aluminum, zirconium, zinc, and mixtures thereof.

8. The antiperspirant application device of claim 7, wherein the antiperspirant active compound comprises a salt of aluminum and zirconium having a ratio of aluminum to zirconium of greater than about 6.0:1.0.

9. The antiperspirant application device of claim 1, wherein the antiperspirant active compound is in a solubilized form.

10. The antiperspirant application device of claim 9, wherein the antiperspirant active compound is solubilized in water.

11. The antiperspirant application device of claim 9, wherein the antiperspirant active compound is solubilized in a monohydric alcohol.

12. The antiperspirant application device of claim 9, wherein the antiperspirant active compound is solubilized in a glycol.

13. The antiperspirant application device of claim 9, wherein the solubilized antiperspirant active compound further comprises one or more of fragrances, dyes, pigments, preservatives, antioxidants, buffering agents, and moisturizers.

14. The antiperspirant application device of claim 9, wherein the solubilized antiperspirant active compound excludes structurants, emollients, and waxes.

15. A method of applying an antiperspirant active compound to the skin comprising the steps of:
applying to the skin an antiperspirant application device comprising a skin-adhesive, transdermal patch comprising an adhesive compound capable of adhesion to human skin and
an antiperspirant active compound based upon an astringent metallic salt disposed within at least a portion of the skin-adhesive, transdermal patch;
leaving the antiperspirant application device in place on the skin for a period of time; and
removing the antiperspirant application device from the skin.

16. The method of claim 15, wherein leaving the antiperspirant application device in place comprises leaving the antiperspirant device in place for a time period of about 1 hour to about 10 hours.

17. The method of claim 16, wherein leaving the antiperspirant application device in place comprises leaving the antiperspirant device in place for a time period of about 2 hours to about 8 hours.

18. The method of claim 17, wherein leaving the antiperspirant application device in place comprises leaving the antiperspirant device in place during a period of sleep.

19. The method of claim 18, wherein removing the antiperspirant application device from the skin comprises removing the antiperspirant application device upon awakening from the period of sleep.

20. The method of claim 15, wherein applying the antiperspirant application device comprises applying the antiperspirant application device to the underarm region of the skin.
